(19) United States
(12) Patent Application Publication
(10) Pub. No.: US 2006/0100607 A1
(43) Pub. Date: May 11, 2006

(54) Catheter insertion aid

(76) Inventor: Tawnya J. Brown, Catonsville, MD (US)

Correspondence Address: ARMSTRONG, KRATZ, QUINTOS,;HANSON & BROOKS, LLP, Suite 220, 502 Washington Avenue, Towson, MD, 21204, US

(21) Appl. No.: 10/984,322

(22) Filed: Nov. 9, 2004

(51) Int. Cl. A61M 27/00

(52) U.S. Cl. 604/544

(57) ABSTRACT

A catheter insertion aid for facilitating urethral insertion of a catheter comprising a hollow device having opposing flexible sides with a slit opening running between said first end and said second end of said device. The slit opening being used for removal of the catheter insertion aid from around the catheter while the catheter is still in place in the vagina. The geometry of the first end of the device being smaller than the second end of the device allowing for easy penetration by the smaller end between the labia folds. The opposing flexible sides of the device when held in a compressed position allow for easy insertion of the device between the labia folds and with the release of the compressed position, the flexible sides expand presenting the urethra for easy insertion of the catheter. A kit containing a catheter insertion aid and a catheter is also disclosed. Alternative embodiments involving a V-shaped tapered device and a device with hinged finger cups and elevations for labia spread are disclosed.

### FIELD OF THE INVENTION

The device of the herein disclosed invention relates to a means for aiding or facilitating the insertion of a medical instrument into a body-orfice.

### BACKGROUND OF THE INVENTION

Urinary elimination is a natural process individuals take for granted until it is altered by some uncontrollable physiologic factor. Patients needing assistance with urinary elimination may require physiologic assistance from skilled personnel. Physiologic support may require the use of an invasive procedure, such as the insertion of a catheter into the bladder. One must be competent in performing this technical skill and sensitive to the patient's psychological needs (Perry and Potter, Clinical Nursing Skills and Techniques, third edition, 1994 (Mosby Publisher), pg. 728).

In many instances where a medical instrument is to be inserted into the urinary orifice, insertion is difficult. The medical device of the herein disclosed invention is designed to facilitate insertion of the medical instrument. Catheterization of female patients is often necessary to remove urine from the bladder for purposes of voiding or for taking a sample. Some patients may require as many as three catheterization procedures over an eight-hour period. Even if the patient has urinated, the doctor may need to order a scan to see if the bladder has emptied. Late stages of a pregnancy may involve multiple catheterizations. Post-surgical patients and those with neurogenic bladder also require catherization. Since urinary retention in the bladder may generate an infection, catheterization to remove urine is very important.

### RELATED PRIOR ART

These cited patents are fairly representative of the prior art.

Prior Art Patents

| Inventor(s) | Patent No. |
|---|---|
| Kobler | 3,054,398 |
| Inoue | 4,200,089 |
| Greene et al | 4,640,273 |
| Woods | 4,782,820 |
| Kaufman | 4,889,107 |
| Yuen | 4,984,564 |
| Koch | 5,851,177 |
| Cosgrove et al | 6,162,172 |
| Butler et al | 6,254,534 |
| Lee | 6,648,819 |

Kobler discloses an eyelid spreader which comprises a short substantially-cylindrical resilient walled tube. The tube is open at both ends. One end has a generally eyeball-shaped contour. This contoured end has diametrically-opposed smoothly-curved recessed portions therein.

Inoue discloses a mouth corner spreader which comprises a pair of oppositely-disposed hooks designed to fit the contour of the patient's mouth. Each hook comprises a substantially C-shaped external position in spaced-apart relationship with a substantially C-shaped internal portion. This internal portion is slightly wider than the external portion and extends outwardly thereof. The internal and external portions are joined by an arcuate portion. A U-shaped spring arm is connected to the lower parts of the respective external portions of both hooks and extends outside of the patient's mouth when the hooks are in place. The extending internal portions have tapered surfaces which flare away from the external portions and engage the inside of the mouth adjacent to the corners.

Greene et al discloses an annular, disposable mouth guard for use with a diagnostic instrument. This mouth guard has a relatively-hard rigid core forward with an opening to permit passage of a diagnostic instrument therethrough. It further comprises a rigid annular center portion (adapted to be held by the patient's teeth), first and second annular end portions, respectively, and a relatively-soft plastic coating which is formed on the radial outer surface of the core to which the patients bite.

Woods discloses an iris retaining device for use during eye surgery. It has an elongated, arcuate, flexible resilient body sized to fit within the inner defining margin of the iris of the human eye. This body includes an iris-receiving sidewall which is generally U-shaped (in cross section) and presents an inboard central bight portion and a pair of spaced-apart wall sections, respectively, extending outwardly of the bight portion. The sidewall is configured for engaging the inner margin of the iris in order to maintain the pupillary opening of a size so as to permit surgical procedures.

Kaufman discloses a surgical retractor which forms a surgical dam for retaining viscera in an abdominal cavity during surgery. The dam is formed by a barrier which substantially resists blood absorption and is non-absorbent on its outer surfaces, with the barrier enclosing a volume of compressible material which is capable of being bent to a selected configuration in a plane perpendicular to the plane of the barrier. The barrier has a plurality of spaced slots for facilitating bends in that plane. Within the compressible material, there is a composite core which bends with the barrier. This core has an “H” configuration and is made of metal (without any “memory”). The overall result is a surgical retractor which is capable of conforming to a spread-apart incision wall, thereby retaining flesh, membrane, muscle and/or viscera.

Yuen discloses a surgical retractor device comprising an inflatable sleeve which, when deflated, is in a pre-coiled position; and when inflated, is uncoiled to define a generally circular aperture.

Koch discloses otoscopes and rhinoscopes, comprising a resilient, flexible deformable ring with plastic memory and a spaced-apart pair of spreaders secured to opposite sides of the ring. Each spreader comprises a transversely-extending support bar bearing a forwardly-projecting spreader blade. When the ring is compressed vertically between the thumb and forefinger, the ring becomes oval shaped and increases the spreading between the blades for widening the field of view through an ear canal or nostril of a patient.

Cosgrove et al discloses a surgical apparatus for retracting animal tissue. The apparatus comprises a resilient, deformable member in the shape of a loop and defining interior and exterior surfaces, respectively. The member is deformable about a region thereof having less resiliency than other regions of the member. In a fully compressed insertion position, opposing regions of the internal surface contact each other. The member also has an expanded tissue-supporting position.

Butler et al discloses a surgical wound retractor, comprising a distal anchoring member for insertion into a wound opening. This distal anchoring member is mounted to a wound-engaging portion of a connecting means. This inner wound-engaging portion of the connecting means has a reduced radial dimension and a retracting configuration, and with the connecting means further having an outer portion with an external guide which is movable relative to the connecting means to shorten the axial extent of the connecting means and thereby bias its wound-engaging portion into a position to retract the wound opening laterally. To maintain retraction of the opening, the connecting means is anchored by an external anchoring means.

Lee discloses a pupil dilator comprising a discontinuous resilient ring adapted to be temporarily straightened linearly thereof to facilitate insertion into, or removal from, an anterior chamber of the patient's eye. The ring has a first end provided with an aperture for engagement by an applicator, and a second end provided with a lip for sliding along the periphery of the pupillary opening in an iris of the eye.

The following patents are exemplary of vaginal speculums. These patents, while allowing access to the vagina, are structurally distinct from the device of the herein invention.

| | |
|---|---|
| Diokno | U.S. 6,569,091 |
| Thompson | U.S. 6,589,168 |
| Nieto | U.S. 6,595,917 |

None of the prior art references, taken singly or in combination, appears to anticipate or renders obvious the structural features and advantages of the disclosed invention.

### OBJECTS OF THE INVENTION

The main object of this invention is to produce a means for efficiently introducing a catheter into the urinary orifice and thus into the bladder.

A further object of this invention is to produce a means which will allow for a less painful administration of a urinary catheter.

A significant object of this invention is to produce a means which allows for efficient urinary catheterization to obtain urine from the bladder.

These and other objects of the present invention will become apparent from a reading of the following specification taken in conjunction with the enclosed drawings.

### BRIEF SUMMARY OF THE INVENTION

The catheter insertion aid of this invention is a pliable plastic accessory that aids in the catheterization of females. Females at a glance are usually more challenging to vaginal catheterization because of their anatomy. The labia folds can often hide the urethra and become very slippery and difficult to manipulate after being swabbed with antiseptic for sterilization. Frequently, manipulation of the inner and outer labia leaves the patient's delicate tissues irritated and sore. Difficulty in visualizing the urethra, particularly in older or obese patients is often a major reason for the catheterization procedure which may be attempted and repeated more than once before success is achieved. In these difficult cases the catheter insertion aid becomes a most helpful tool. The dexterous nature of the inventive appliance aids in applying pressure to the labia in a more uniformed fashion than by simply using the fingertips. Accordingly, the one-hand held two-sided insertion aid of this invention can be used to manipulate the folds of the labia up or down, left or right with substantially less discomfort to the patient; and thus allowing for facile insertion of the catheter. For smaller labial folds, the smaller end of the device can be used and the larger end can be used for larger labial folds. The hollow character of the device allows for a clearer visualization of the urethra thus increasing the accuracy and success of catheter placement.

Broadly considered, the invention is directed to a catheter insertion aid for facilitating urethral insertion of a catheter comprising a pliable bifurcated device flexibly hinged at the point of bifurcation such that opposite sides of the device when compressed will allow the device to be inserted between the labia folds and when compression is released from opposite sides of the device the labia folds are spread allowing the urethra to be presented for urethral catheterization.

The device of the herein disclosed invention is intended to provide a means for quickly and conveniently catheterizing a female patient with the intention of removing urine from the bladder. The insertion aid is a spring-like, flexible, pliable, molded device used to spread the lips of the labial folds. Small elevations on the outer diameter of the catheter insertion aid promote easy griping and handling. In use the device is slightly compressed by gripping it manually between the thumb and fingers and placed within the labial folds. When in place, the grip on the device is loosened allowing the diameter of the device to spread wider, allowing the labia folds to follow. The catheter is then inserted through the catheter insertion aid. The device allocates uniform pressure to be applied to the labia of the patient's vagina; and allows for a substantially clearer visualization of the urethra. With the urethra in plain view, the catheter can be readily guided into the urinary opening and then into the bladder at which point the insertion aid can be removed from around the catheter. Once catheterization is completed, the catheter can be left in place or can be removed from the bladder and urethra as circumstances dictate. The beauty of the catheter insertion aid is that it is designed with a longitudinal split-opening along its side which allows the aid to be removed from the catheter while one end of the catheter is in place in the urethra and the other end attached to the urine receiving receptacle. The insertion aid once removed may be discarded into an appropriate receptacle. Disposal is convenient since the insertion aiding device is relatively inexpensive to make and thus is conveniently disposable.

As an alternative embodiment, while not preferred the device would not have to be tapered, but could have the top end and bottom end of approximately the same dimensions.

The catheter insertion aid has the following characteristics:

1. The sides of the device are to be roughened (almost like a knurl) to provide a better grip or else a series of spread-apart protrusions are used to achieve the same result. There will not be the need for concavities or finger indents.

2. The device is tapered for easier insertion and reversible for young girls. Once the aid hits the bone, it stops.

3. The device is to be molded from a suitable (“rubberized material or plastic”) which, preferably, would be non-allergic.

4. The device is provided with a longitudinal side slit or opening which allows the insertion aid to slip over and away from the catheter for removal, while the catheter stays in place.

5. The walls of the device are thin spring-like and flexible and will yield to finger pressure. There will be more space inside and the rim is shorter. The device is generally oval or elliptical in cross-section. And is tapered (somewhat conical); in addition, the wall thickness decreases in the direction of the taper.

6. The device will allow for the viewing of the urethra which is often difficult in older or obese patients.

### BRIEF DESCRIPTION OF THE DRAWINGS

FIG. 1 is an illustration depicting the prior art method of inserting a urinary catheter.

FIG. 2 is a perspective view of the catheter insertion aid of the herein disclosed invention.

FIG. 3 is a cross-sectional view thereof taken along lines 3-3 of FIG. 2.

FIG. 4 is a top plan view thereof.

FIG. 5 is a bottom plan view thereof.

FIG. 6 is a pictorial view of the catheter insertion aid prior to being placed between the labia folds.

FIG. 7 is a pictorial view of the catheter insertion aid inserted between the labia folds.

FIG. 8 is a pictorial view of the catheter about to be inserted into the urethra through the catheter insertion aid.

FIG. 9 is a pictorial view of the catheter being inserted through the catheter insertion aid.

FIG. 10 is a pictorial view of the catheter in place and the catheter insertion aid being removed from the vaginal folds and from around the catheter.

FIG. 11 is a view illustrating the catheter in place with the insertion aid being easily disposed of.

FIG. 12 is a kit containing a catheter insertion aid along with a catheter.

FIG. 13 illustrates a catheter insertion aid wherein the first end measures substantially the same as the second end.

FIG. 14 is a cross-section thereof taken along line 14-14 of FIG. 13.

FIG. 15A and 15B are perspective views of an alternative embodiment of the catheter insertion aid in a V-shaped tapered configuration. FIG. 15A illustrates the device in a compressed state and FIG. 1 5B illustrates the device in a relaxed state.

FIG. 15C is a rear plan view of the alternative embodiment catheter insertion aid of the V-shaped tapered configuration in the relaxed state.

FIG. 15D is a front plan view thereof.

FIG. 16A is a perspective view of a further alternative embodiment of a catheter insertion aid having finger-cups and elevations on the front or forward sides of the finger cups. Fingers are shown inserted in the finger cups.

FIG. 16B is a side plan view thereof.

FIG. 16C is a front plan view thereof in the relaxed state.

FIG. 16D is a front plan view thereof in the compressed state.

FIG. 16E is a side plan view thereof.

FIG. 16F is a cross-sectional view taken off of 16F - - - 16F of FIG. 16E.

FIG. 16G is a view thereof in the compressed state prior to insertion.

FIG. 16H is a pictorial view thereof inserted between the labia folds in the compressed state.

FIG. 161 is a view thereof inserted between the labia folds in the relaxed or expanded state.

FIG. 17 is a block-diagram flowchart, showing a method of using the catheter insertion aid.

### DESCRIPTION OF THE INVENTION

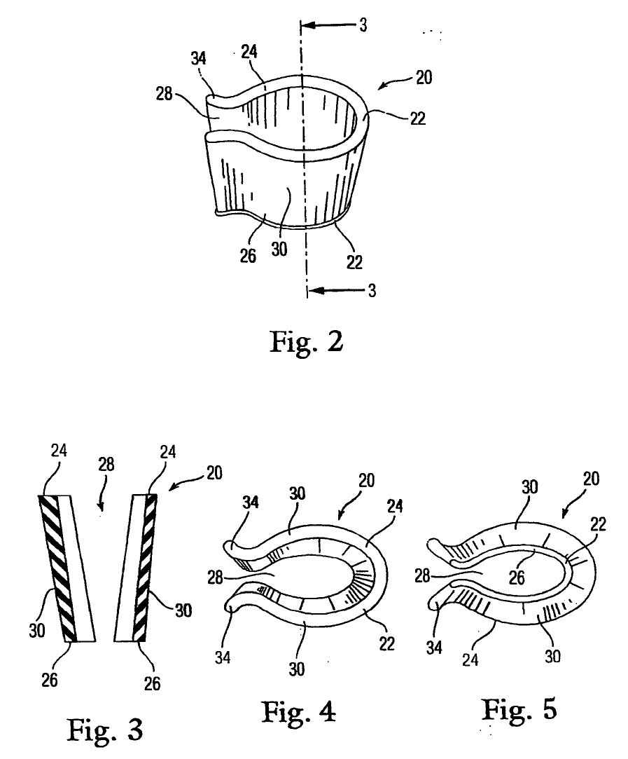

The catheter insertion aid 20 (FIG. 2) is used after sterile preparation of the labia related areas with an antiseptic. Either side of the device 20 may be used depending on the size of the labia. In use the device is slightly compressed by the thumb and fingers and wedged between the labia folds. Lightening the grip on the device once it is in place allows the labia to expand revealing the urethra for catheter placement. Once catheterization has taken place, the split design of the aid allows for easy removal from around the catheter while leaving the catheter in place.

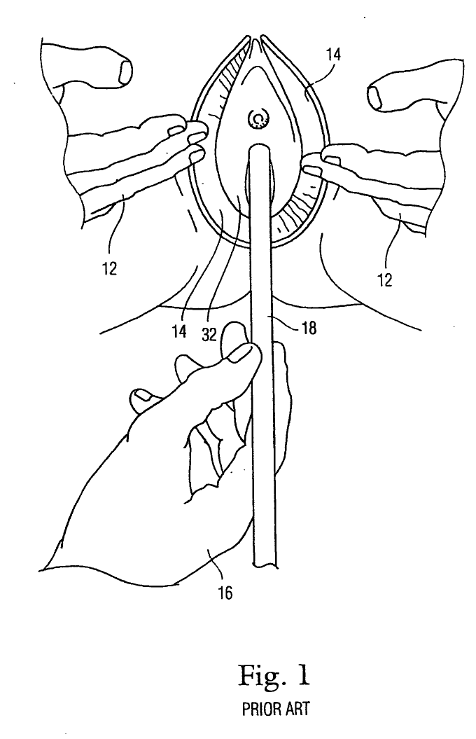

With reference to FIG. 1, the prior art method for inserting a catheter 18 frequently involves a first technician using two hands 12 to spread the labial folds 14 and a second technician 16 for inserting the catheter 18. In other words the prior art vaginal catheter insertion was a two-person operation.

With reference to FIG. 2 the catheter insertion aid 20 when viewed from the top or bottom has a horseshoe-like configuration 22. The aid further has a first end 24 and a second end 26 with a slit-like opening 28 running from the first end to the second end.

Referring to cross-section FIG. 3 note that the device has a tapered configuration 30 designed to allow for easy access to the vagina.

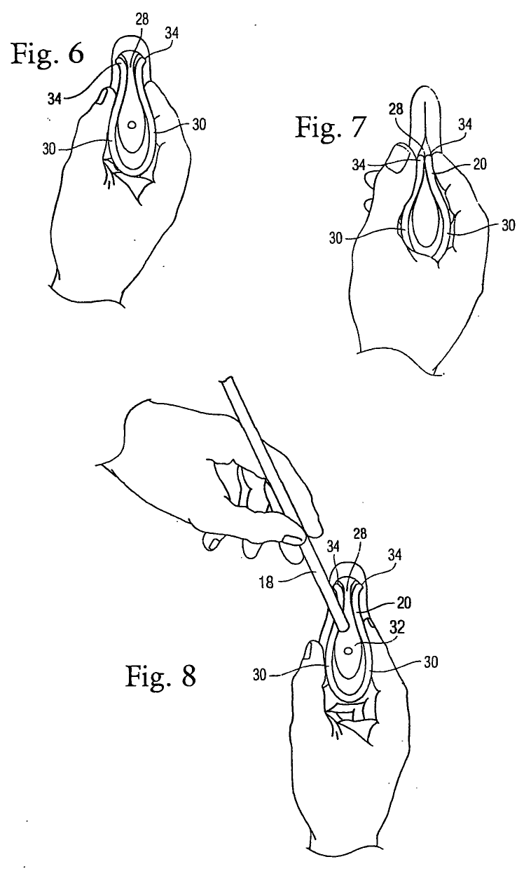

FIG. 4 is a top plan view of the catheter insertion aid and FIG. 5 is a bottom plan view thereof. Note particularly that the sides of the insertion aid are flexible and are able to yield to finger pressure for easy insertion between the labia folds 32. After insertion, the grip is lessened and the sides of the device expand spreading the labial folds allowing for easy access and insertion of the catheter 18. Note that finger pressure on sides 30 causes slit-opening 28 to narrow in a pincer-like manner and release of finger pressure on sides 30 causes the sides of the insertion aid to expand allowing for easy insertion of the catheter into the urethra Note also that in FIG. 6 tabs 34 forming slit-opening 28 are spread apart; this is the relaxed state. For insertion, the sides are compressed and the slit-opening 28 is closed (FIG. 7). Once the insertion aid is in place and finger pressure released, the tabs are spread apart and the slit-opening assumes the relaxed or open position.

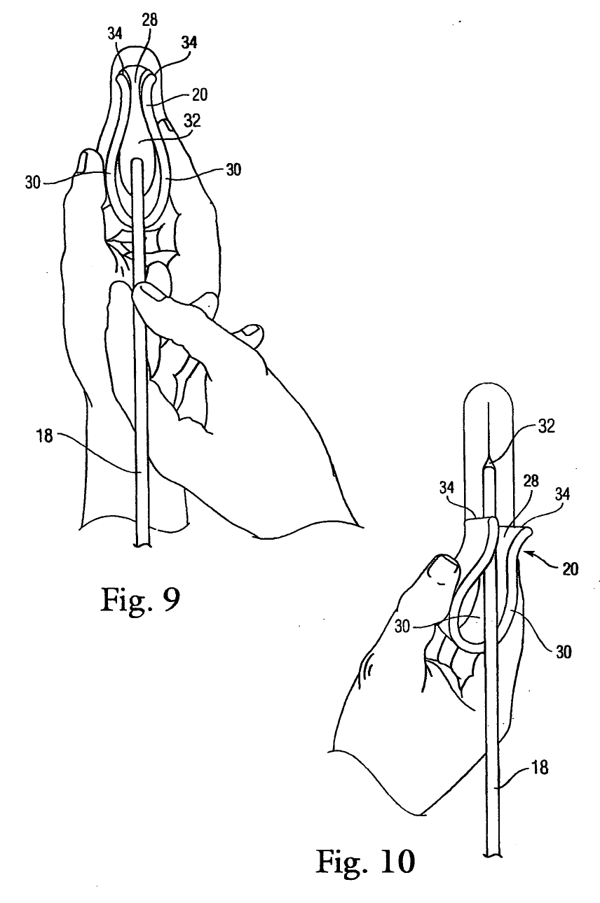

FIGS. 6-10 describe how the catheter insertion aid 20 is to be used to allow for easy insertion of a catheter into the urethra. In FIG. 6 the sides of the aid 30 are grasped and then are squeezed together by finger pressure (FIG. 7) for insertion between the labia folds 32. Once inserted into the urethra (FIG. 7), finger pressure on the sides 30 is released spreading the labia folds (FIG. 8). With finger release, aid 20 expands to its unflexed state and the catheter 18 is ready to be inserted into the urethra 32 through the insertion aid 20 (FIG. 10). With the catheter 18 properly positioned (FIG. 9) the insertion aid can be removed through slit-opening 28 (FIG. 10).

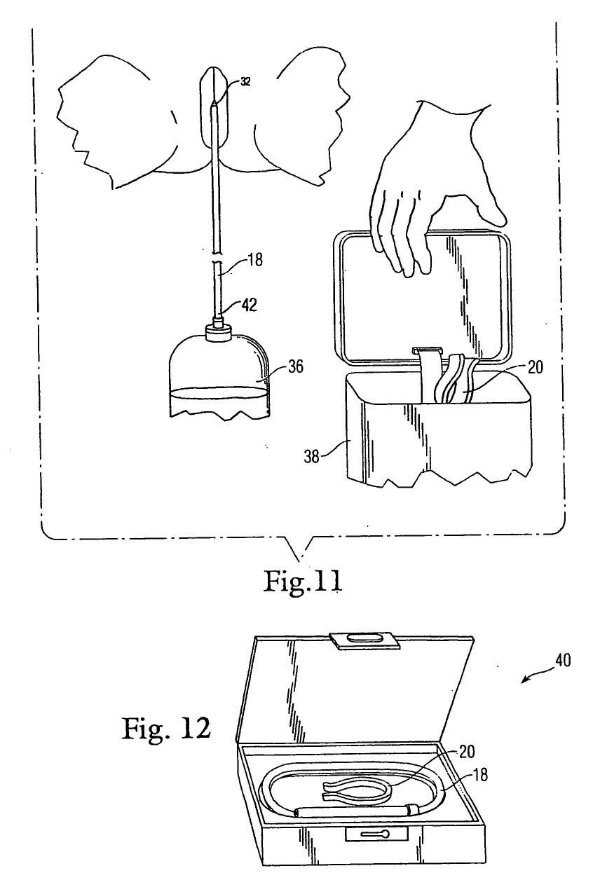

FIG. 11 describes the easy disposal of the catheter insertion aid 20 into a receptacle 36 while one end of the catheter is in place and the other end is attached to a receptacle for receiving urine.

FIG. 12 illustrates a kit 40 containing therein the catheter insertion aid 20 and a catheter 18.

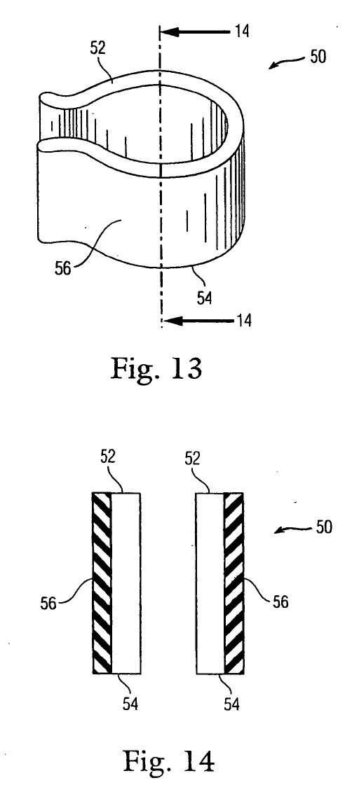

FIG. 13 illustrates an alternative embodiment of a catheter insertion aid 50 wherein the first end 52 measures substantially the same as the second end 54; and wherein the sides thereof 56 are substantially parallel.

FIG. 14 is a cross-section thereof taken along line 14-14.

With reference to FIGS. 1-14, the dimensions of the catheter insertion aid will vary depending on the patient. Specifically for a young patient the dimensions will be 1½ to 2 inches in width and 2 inches in height, of course, dimensions will vary depending on the patient's age, size and weight.

### Alternate Embodiments

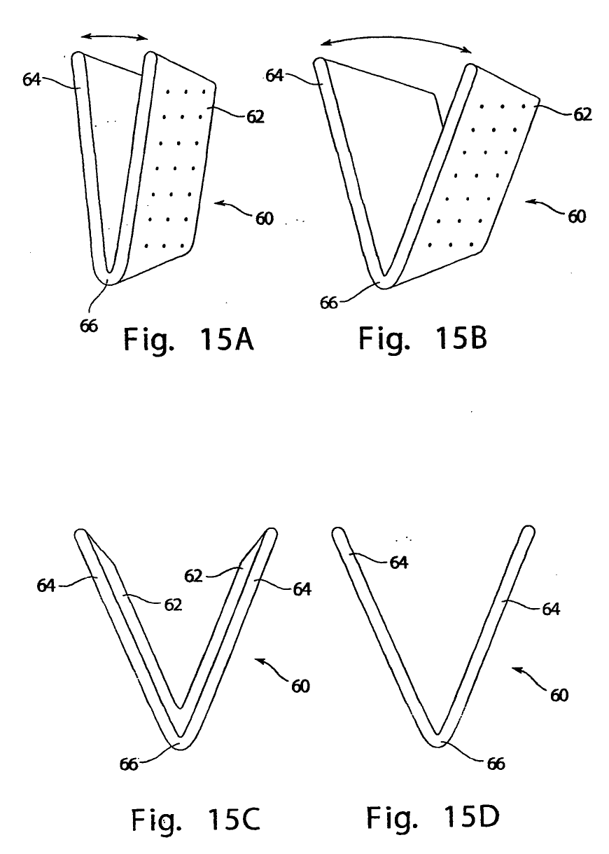

A relatively simple embodiment of a catheter insertion aid 60 is shown in FIGS. 15A and 15B and involves a tapered V-shaped device 60 having a front end 62 and a rear end 64 and a flexible apex 66 which flexes to allow the device to be compressed or closed FIG. 15A or relaxed or opened FIGS. 15B, 15C and 15D. In use the device is compressed using the thumb and fingers FIG. 15A and is inserted between the labia folds. Once inserted, the thumb and fingers are removed from the device 60 and the catheter insertion aid assumes the relaxed or expanded state FIG. 15B expanding the labia folds and allowing for easy access for insertion of a catheter into the urethra. Once the catheter has been inserted, the catheter insertion aid 60 can be removed from the vagina and from around the catheter while the catheter remains in place to receive urine. The catheter insertion aid once removed can be discarded.

The tapered V-shaped catheter insertion aid 60 can be made of a semi-pliable, clear material and may have elevated grips or projections to make handling easier. The dimensions of the tapered V-shaped device 60 can vary depending upon the size of the patient; however, dimensions of a representative device would be 2½ inches in height and 1½ inches in width. The material from which the device is made has a thickness of approximately 3/16 of an inch and may be made of pre-shaped plastic having a memory to return to the relaxed position.

While the catheter insertion aid has been described as being in a tapered configuration, it is obvious that other configurations would be operative. For example, the device could have a cylindrical configuration. Further, the cylindrical device could be stream-lined to enhance insertion capability.

It is to be noted that particularly the catheter insertion aid described in FIGS. 2-5 and FIGS. 15A and B could have the leading edges thereof lubricated for easier insertion between the labia folds.

### Further Alternate Embodiment

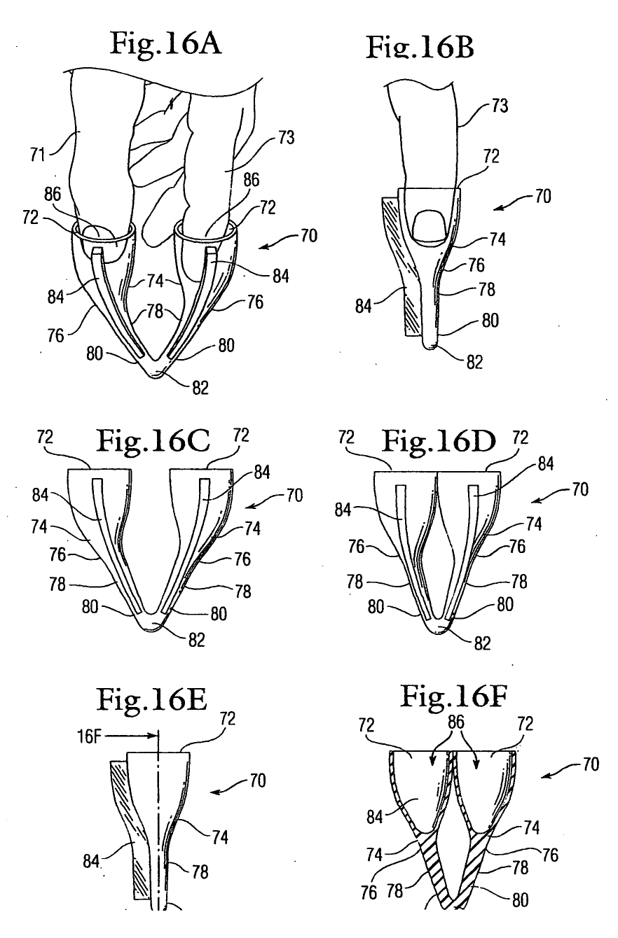

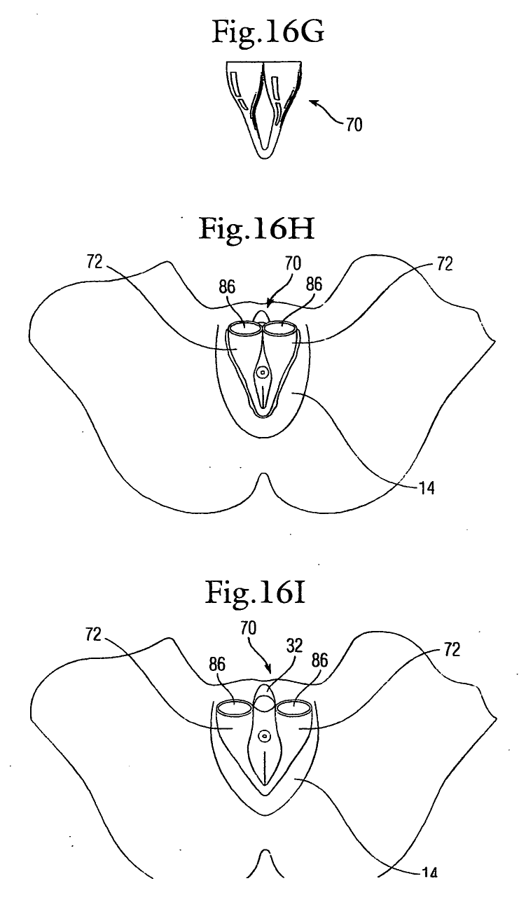

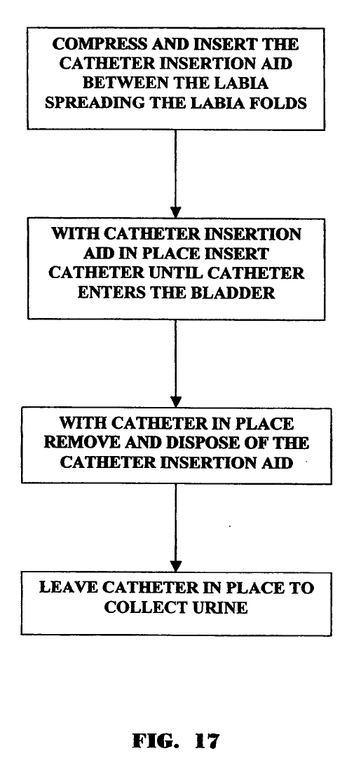

Referring to FIG. 16A through FIG. 16I an elegant alternate catheter insertion aid 70 has a pair of finger cups 72, which are tulip-shaped (however, other configurations are possible). Joined to the base 74 of each finger cup 72 is the first end 76 of stem 78 and the second end 80 of stem 78. The second end 80 of stems 78 are joined together to form a V-shaped spring hinge 82. The V-shaped spring hinge 82 has a compressed configuration (FIGS. 16D and 16F) and a relaxed configuration (FIGS. 16A and 16C). Note that attached to the front side of finger cup 72 and stem 78 is elevation or elevated projection 84.

Referring specifically to FIG. 16A and FIG. 16C, in use the thumb and index fingers are inserted into openings 86 of the finger cup 72 while catheter insertion aid 70 is in the relaxed position (FIGS. 16A and D). The thumb and index finger are brought together bringing the finger cups 72 together (FIG. 16D and FIG. 16G). While the aid 70 is in the compressed state, elevations 84 are inserted between the labia folds (FIG. 16H) and once the inserted fingers are released to bring aid 70 to its relaxed state (FIG. 16I). In the relaxed state the labia folds are spread apart allowing for access of the catheter into the urethra.

With reference to FIGS. 16A and 16B, in use the thumb 71 and index finger 73 are each inserted into a single finger cup 72 and the fingers compressed to cause the V-shaped spring hinge 82 to load or compress and bring the elevations 84 on the finger cups 72 and stems to approach each other (FIG. 16D and FIGS. 16G). The elevations or elevated projections 84 on the forward portion of the finger cup and stem are inserted between the labia folds (FIG. 16H) and then the fingers within the cups 72 are released (FIGS. 16C and 16I) to spread the labia folds to expose the urethra for easy insertion of a catheter. Once the catheter is in place, the catheter insertion aid can be removed from the vagina and from around the catheter and the catheter insertion aid discarded. The elevated projections 84 projecting longitudinally from the front or forward portion of each finger cup 72 and along each stem 78 are approximately a quarter inch in elevation and running longitudinally approximately 2½ inches. The opening 86 in the finger cup 72 for finger insertion is approximately ¾ inches across.

FIG. 17 is a flow-diagram illustrating the process for using the catheter insertion aid.

The herein disclosed catheter insertion aid has been described as being flexibly hinged at the apex to allow for compression and relaxation. The flexible hinge could be made of plastic with a memory to return to its non-compressed state. Another embodiment would involve a mechanical spring arrangement at the flexible hinge allowing for compression and relaxation at the hinged apex.

### ADVANTAGES OF THE INVENTION

A most important advantage of the herein disclosed invention is that without the use of the catheter insertion aid, insertion would be a three-handed” two-person operation. For example, the technician would employ both hands to push the labia open, and a second technician would use a hand to insert the catheter into the urethra. Using the device and method of this invention, a single nurse or technician can hold the device with one hand to hold the labia apart, and use the other hand to connect the catheter to the urethra. Thus, the need for a second person being with the patient during catheterization is eliminated.

The device makes catheterization easier and increases the accuracy of hitting the mark after a first attempt, because better visualization means fewer failed attempts to carry out the catheterization process. This in turn means less handling and irritation to delicate tissues along with less patient discomfort.

Increased visualization of urethra and reduced irritation because of less manipulation of delicate tissues is an added benefit.

Flexible material and dexterous design allows for easy use.

The longitudinal split opening on the catheter insertion aid allows for removal of the insertion aid while one end of the catheter is urethrally inserted and the second end attached to the urine receiving receptacle.

The device is inexpensive to make and is therefore conveniently disposable.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A catheter insertion aid for facilitating urethral insertion of a catheter comprising a pliable bifurcated device flexibly hinged at the point of bifurcation such that opposite sides of the device when compressed will allow the device to be inserted between the labia folds and when compression is released from opposite sides of the device the labia folds are spread allowing the urethra to be presented for urethral catheterization.

2. A catheter insertion aid for facilitating urethral insertion of a catheter comprising a hollow device having opposing flexible sides and also having a first end and a second end with a slit opening running between said first end and said second end with said slit opening to be used for removal of the catheter insertion aid from around the catheter and wherein the geometry of the first end of the device is smaller than the second end of the device allowing for easy penetration by the smaller end of the device between the labia folds, with said opposing flexible sides of the device held in a compressed position allowing for easy insertion of the device between the labia folds and with the release from the compressed position, the flexible sides expand presenting the urethra for insertion of the catheter.

3. The catheter insertion aid of claim 2 having a horseshoe-like configuration when viewed from the first end.

4. The catheter insertion aid of claim 2 further having a lubricant applied to the smaller first end of the catheter insertion aid in order to facilitate insertion.

5. The catheter insertion aid of claim 2 in combination with a catheter wherein the catheter insertion aid facilitates the insertion of the catheter into the urethra.

6. A catheter insertion aid for facilitating urethral insertion of a catheter comprising a hollow device having opposing flexible sides and also having a first end and a second end with a slit opening running between said first end and said second end with said slit opening to be used for removal of the catheter insertion aid from around the catheter and wherein the dimensions of the first end of the device is the same as that of the second end of the device with the first end of the device being able to penetrate between the labia folds with said opposing flexible sides of the device held in a compressed position allowing for easy insertion of the device between the labia folds and with the release from the compressed position, the flexible sides expand presenting the urethra for insertion of the catheter.

7. A method for facilitating the insertion of a catheter into the urethra comprising the steps of:
a.) inserting the catheter insertion aid of claim 1 between the labia while grasping and compressing the flexible sides,
b.) releasing the flexible sides of the catheter insertion aid,
c.) inserting a catheter into the urethra through the catheter insertion aid and into the bladder and
d.) removing the catheter insertion aid through the slit opening of the catheter insertion aid.

8. A method for quickly and conveniently catherizing a female patient for purposes of urinary elimination, comprising the steps of providing a pliable molded hollow device, gripping the device manually and inserting the device between the labia folds of the vagina thus manipulating the folds of the labia up or down, left or right, with substantially less discomfort to the patient, maintaining the manual grip on the device with one hand as the device exerts substantially uniform pressure on the labia of the patient's vagina, the device allowing for a substantially clearer visualization of the urethra, providing a catheter, passing the catheter through the device to connect the catheter to the patient's bladder, and removing said device from the catheter while the catheter remains in the patient.

9. The method of claim 8, further comprising the step of disposing of the device following its removal from the patient while the catheter stays in place.

10. A kit comprising a package containing therein a catheter insertion aid along with a catheter.

11. A catheter insertion aid comprising a device having tapered sides with the sides forming a V-shaped configuration and with the apex of the V-shaped configured device being spring-like allowing the sides of the device to be close together in the compressed state and allowing the sides to expand when the device is in the relaxed state and with the smaller end of the tapered sides allowing for easy penetration of the catheter insertion aid between the labia folds of the vagina, so that the insertion of a catheter into the urethra can be facilitated.

12. A method of facilitating the insertion of a catheter into the urethra comprising the steps of:
a.) inserting the catheter insertion aid of claim 11 between the labia while grasping and compressing the flexible sides,
b.) releasing the flexible sides of the catheter insertion aid,
c.) inserting a catheter into the urethra through the catheter insertion aid and into the bladder and
d.) removing the catheter insertion aid through the slit opening of the catheter insertion aid.

13. A catheter insertion aid comprising a pair of finger cups supported on a pair of stems, the pair of stems joining to form a V-shaped hinge, the V-shaped hinge being spring-like thus allowing the device to be either compressed or relaxed, the finger cups and attached stems each having a front side provided with a pair of longitudinal elevations, compressing the fingers in the finger cups causes the cups and longitudinal elevations thereon to be brought together allowing the catheter insertion aid to be inserted between the labia fold of the vagina; and by releasing the fingers, catheter insertion aid relaxes spreading the labia folds to enable insertion of a catheter into the urethra.

14. A method for facilitating the insertion of a catheter into the urethra comprising the steps of:
a.) inserting the catheter insertion aid of claim 13 between the labia by grasping and compressing the finger cups to bring together the longitudinal elevations and inserting said longitudinal elevations between the labia folds,
b.) releasing the finger cups of the catheter insertion aid thereby allowing the device to relax and expand spreading the labia folds to present the urethra,
c.) inserting a catheter into the urethra through the catheter insertion aid and into the bladder and
d.) removing the catheter insertion aid through the slit opening of the catheter insertion aid.
